(19) United States
(12) Patent Application Publication
(10) Pub. No.: US 2026/0325737 A1
(43) Pub. Date: Oct. 22, 2026

(54) ENCAPSULATED NEGATIVE PRESSURE AND WOUND PRESSURE SENSING DEVICES

(71) Applicant: SOLVENTUM INTELLECTUAL PROPERTIES COMPANY, Maplewood, MN (US)

(72) Inventors: Benjamin A. PRATT, Poole (GB); Robert HOWARD, Westmeath (GB); Dominic NOLAN, Bracknell (GB)

(21) Appl. No.: 19/104,340

(22) PCT Filed: Jul. 19, 2023
(86) PCT No.: PCT/IB2023/057354

§ 371 Date: Feb. 17, 2025

Related U.S. Application Data
(60) Provisional application No. 63/402,340, filed on Aug. 30, 2022.

(51) Int. Cl. A61M 1/00

(52) U.S. Cl. CPC A61M 1/912; A61M 1/962; A61M 1/964; A61M 1/966; A61M 2205/3344; A61M 2205/502; A61M 2205/75; A61M 2205/82; A61M 2207/00

(57) ABSTRACT

A system for treating a tissue site with negative pressure. The system includes a dressing, an electric circuit, a cover, an encapsulant, a negative pressure source, and a tube. The dressing is configured to be disposed at a tissue site. The electric circuit includes a power source and at least one sensor. The cover includes at least one cavity and a negative pressure port. The at least one cavity is configured to create a sealed enclosure around the at least one sensor. The encapsulant surrounds the cover and at least a portion of the electric circuit. The tube is configured to be fluidly coupled to the negative pressure port and to communicate negative pressure between the negative pressure source and the dressing. Other related systems, devices, and methods are also disclosed.

### CROSS-REFERENCE TO RELATED APPLICATIONS

This application is a U.S. National Stage Entry of International Application No. PCT/IB2023/057354, filed Jul. 19, 2023, which claims the benefit of priority to U.S. Provisional Application No. 63/402,340, filed on Aug. 30, 2022, each of which are incorporated herein by reference in their entirety.

### TECHNICAL FIELD

The invention set forth in the appended claims relates generally to tissue treatment systems and more particularly, but without limitation, to encapsulated negative pressure and wound pressure sensing devices.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for encapsulating negative pressure and wound pressure sensing devices in a negative-pressure therapy environment are set forth in the appended claims. Illustrative embodiments are also provided to enable a person skilled in the art to make and use the claimed subject matter.

For example, in some embodiments, a system for treating a tissue site with negative pressure is described. In some example embodiments, the system can include a dressing, an electric circuit, a cover, an encapsulant, a negative pressure source, and a tube. The dressing can be configured to be disposed at a tissue site. The electric circuit can include a power source and at least one sensor. The cover can include at least one cavity and a negative pressure port. The at least one cavity can be configured to create a sealed enclosure around the at least one sensor. The encapsulant can surround the cover and at least a portion of the electric circuit. The tube can be configured to be fluidly coupled to the negative pressure port and to communicate negative pressure between the negative pressure source and the dressing.

In some example embodiments, the cover can further include a negative pressure cavity in fluid communication with the negative pressure port. The negative pressure cavity can be configured to be positioned in fluid communication with the dressing. In some example embodiments, the cover can further include a sensing cavity configured to be fluidly isolated from the negative pressure cavity. The sensing cavity can be configured to create the sealed enclosure around the at least one sensor.

In some example embodiments, the at least one sensor can be a pressure sensor.

In some example embodiments, the electric circuit can include a printed circuit board. The cover can be configured to create the sealed enclosure relative to the printed circuit board. In some example embodiments, the printed circuit board can include a rigid material and the encapsulant can include a flexible material. In some example embodiments, the printed circuit board can be formed of a material that is more rigid than the encapsulant.

In some example embodiments, the at least one cavity can be a negative pressure cavity. The negative pressure cavity can be configured to create the sealed enclosure around the at least one sensor. The negative pressure cavity can be in fluid communication with the negative pressure port and can be configured to be positioned in fluid communication with the dressing.

In some example embodiments, the electric circuit can include the negative pressure source. The at least one sensor can include a negative pressure sensor and an ambient pressure sensor. The cover can be configured to create the sealed enclosure around the negative pressure source and the negative pressure sensor. In some example embodiments, the sealed enclosure can be a first sealed enclosure. The cover can be configured to create a second sealed enclosure configured to enclose the ambient pressure sensor separate from the first sealed enclosure.

In some example embodiments, the cover can include a first exhaust aperture and a first ambient pressure sensor aperture. The first exhaust aperture can be configured to provide a path for exhaust from the negative pressure source to escape through the cover. The first ambient pressure sensor aperture can be configured to provide a path from the ambient pressure sensor to an ambient environment surrounding the system.

In some example embodiments, the encapsulant can include a second exhaust aperture and a second ambient pressure sensor aperture. The second exhaust aperture can be configured to provide a path for exhaust from the negative pressure source to escape through the first exhaust aperture and the encapsulant. The second ambient pressure sensor aperture can be configured to provide a path from the ambient pressure sensor through the first ambient pressure sensor aperture to the ambient environment surrounding the system.

In some example embodiments, the system can further include a first filter, a second filter, a membrane, and an ambient environment pathway. The first filter can be configured to couple with the second exhaust aperture. The second filter can be configured to couple with the second ambient pressure sensor aperture. The membrane can be configured to couple to the encapsulant to enclose the first filter and the second filter between the membrane and the encapsulant. The ambient environment pathway can be on a surface of the encapsulant. The ambient environment pathway can be configured to connect the second exhaust aperture and the second ambient pressure sensor aperture to the ambient environment surrounding the system.

In some example embodiments, the encapsulant can include an ambient pressure sensor aperture and a first exhaust aperture. The ambient pressure sensor aperture can be configured to provide a path from the ambient pressure sensor to an ambient environment surrounding the system. The first exhaust aperture can be configured to align with a second exhaust aperture of the cover. The first exhaust aperture and the second exhaust aperture can be configured to provide a path for exhaust from the negative pressure source to reach the ambient environment surrounding the system.

In some example embodiments, the system can further include a power button configured to actuate the negative pressure source. In some example embodiments, the port button can be embedded within the encapsulant and the encapsulant can be configured to allow a user to depress the power button.

In some example embodiments, the encapsulant can include a power source aperture configured to expose the power source to an ambient environment surrounding the system. In some example embodiments, the at least one sensor can include an ambient pressure sensor disposed proximate to the power source. The ambient pressure sensor and the power source can both be exposed to the ambient environment surrounding the system through the power source aperture.

In some example embodiments, the power source can be coupled to a wireless charging device configured to charge the power source through the encapsulant.

In some example embodiments, the at least one cavity can include a negative pressure cavity and a sensing cavity isolated from the negative pressure cavity. The at least one sensor can include a negative pressure sensor and an ambient pressure sensor. The negative pressure cavity can be configured to create a first sealed enclosure around the negative pressure sensor and the sensing cavity can be configured to create a second sealed enclosure around the ambient pressure sensor. The first sealed enclosure can be fluidly isolated from the second sealed enclosure. The negative pressure cavity can be in fluid communication with the negative pressure port and can be configured to be positioned in fluid communication with the dressing.

In some example embodiments, the at least one sensor can include a negative pressure sensor and an ambient pressure sensor. The at least one cavity of the cover can include a sensing cavity fluidly isolated from a negative pressure cavity. The sensing cavity can be configured to create the sealed enclosure around the negative pressure sensor. The sensing cavity and the sealed enclosure can be fluidly isolated from the ambient pressure sensor. The negative pressure cavity can be in fluid communication with the negative pressure port. In some example embodiments, the cover can further include a sensing aperture and a negative pressure opening. The sensing aperture can be configured to expose the sensing cavity and the negative pressure sensor to the dressing. The negative pressure opening can be configured to provide a path from the negative pressure cavity to the dressing. In some example embodiments, the encapsulant can include an opening configured to couple the sensing aperture and the negative pressure opening to the dressing.

In some example embodiments, the electric circuit further includes at least one indicator. The at least one indicator can be configured to indicate a status of the system.

In some example embodiments, the encapsulant is substantially transparent.

In some example embodiments, the encapsulant is substantially opaque.

Also described herein is a method of manufacturing a device. The method can include providing a printed circuit board including electronic components, coupling a cover around at least one electronic component, and encapsulating the printed circuit board and the cover in an encapsulant. In some example embodiments, the cover can be configured to provide a fluid seal around the at least one electronic component.

In some example embodiments, the method can further include drilling at least one hole through the encapsulant and the cover to provide a fluid path from the at least one electronic component to an exterior of the device.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a block diagram of an example embodiment of a therapy system that can provide negative-pressure treatment in accordance with this specification;

FIG. 2A is an exploded view of an example negative pressure device suitable for use with the therapy system of FIG. 1;

FIG. 2B is a perspective view of the negative pressure device of FIG. 2A;

FIG. 2C is a perspective view of the negative pressure device of FIG. 2B surrounded by an encapsulant;

FIG. 2D is a perspective view of a cover of the negative pressure device of FIG. 2A;

FIG. 2E is a cross-sectional view of the cover of FIG. 2D taken along the line 2E-2E depicted in FIG. 2D;

FIG. 2F is a perspective view of a bottom surface of the cover of FIG. 2D;

FIG. 3A is a top view of another embodiment of a negative pressure device including a power button that may be associated with an example embodiment of the therapy system of FIG. 1;

FIG. 3B is a perspective view of the negative pressure device of FIG. 3A;

FIG. 3C is a perspective view of a cover of the negative pressure device of FIG. 3A;

FIG. 3D is a perspective view of a bottom surface of the cover of FIG. 3C;

FIG. 3E is a cross-sectional view of the cover of FIG. 3C taken along the line 3E-3E depicted in FIG. 3C;

FIG. 4A is a top view of another embodiment of a negative pressure device with a power button that may be associated with an example embodiment of the therapy system of FIG. 1;

FIG. 4B is a side view of the negative pressure device of FIG. 4A taken at line 4B-4B depicted in FIG. 4A;

FIG. 5A is a top view of another embodiment of a negative pressure device with an exposed ambient pressure sensor that may be associated with an example embodiment of the therapy system of FIG. 1;

FIG. 5B is a perspective view of the negative pressure device of FIG. 5A;

FIG. 6 is a perspective view of another embodiment of a negative pressure device with an exposed power source that may be associated with an example embodiment of the therapy system of FIG. 1;

FIG. 7 is a perspective view of another embodiment of a negative pressure device with an exposed power source and ambient pressure sensor that may be associated with an example embodiment of the therapy system of FIG. 1;

FIG. 8 is a perspective view of another embodiment of a negative pressure device with a charging coil that may be associated with an example embodiment of the therapy system of FIG. 1;

FIG. 9A is an exploded view of another example negative pressure device that may be associated with an example embodiment of the therapy system of FIG. 1;

FIG. 9B is an exploded view of the negative pressure device of FIG. 9A surrounded by an encapsulant;

FIG. 9C is a perspective view of the negative pressure device of FIG. 9B;

FIG. 10A is an exploded view of a wound pressure sensing device that may be associated with an example embodiment of the therapy system of FIG. 1;

FIG. 10B is a side, cut-away view of the wound pressure sensing device of FIG. 10A;

FIG. 10C is a side, cut-away view of the wound pressure sensing device of FIG. 10A surrounded by an encapsulant;

FIG. 10D is a side, cut-away view of the wound pressure sensing device of FIG. 10C with at least one hole drilled through the encapsulant and the cover;

FIG. 10E is a perspective view of the wound pressure sensing device of FIG. 10D;

FIG. 11A is an exploded view of another example wound pressure sensing device that may be associated with an example embodiment of the therapy system of FIG. 1;

FIG. 11B is a side, cut-away view of the wound pressure sensing device of FIG. 11A;

FIG. 11C is a side, cut-away view of the wound pressure sensing device of FIG. 11A surrounded by an encapsulant; and

FIG. 11D is a perspective view of the wound pressure sensing device of FIG. 11C.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

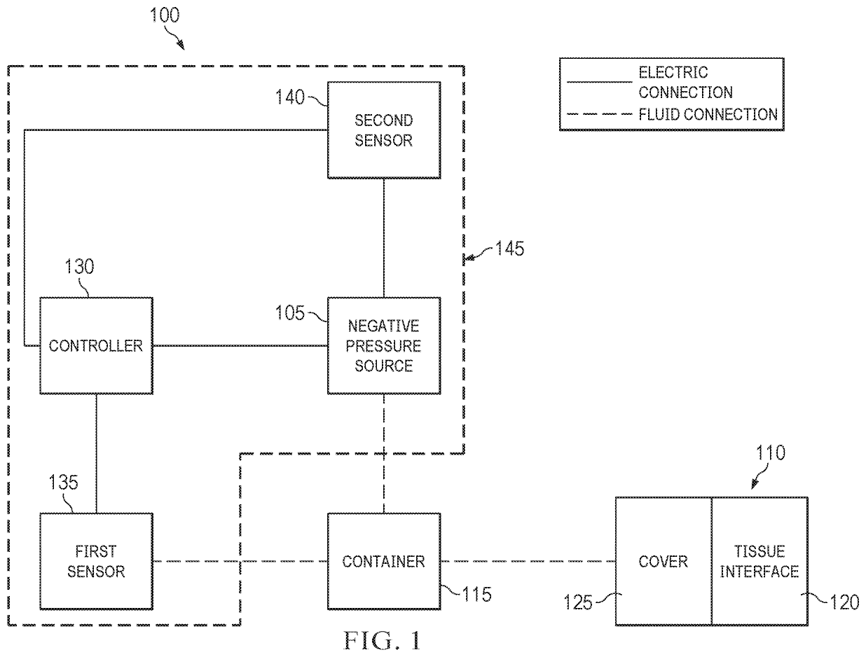

FIG. 1 is a block diagram of an example embodiment of a therapy system 100 that can provide negative-pressure therapy to a tissue site in accordance with this specification.

The therapy system 100 may include a source or supply of negative pressure, such as a negative pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from 3M Company.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

Some components of the therapy system 100 may be housed within or used in conjunction with other components, such as sensors, processing units, alarm indicators, memory, databases, software, display devices, or user interfaces that further facilitate therapy. For example, in some embodiments, the negative pressure source 105 may be combined with the controller 130 and other components into a therapy unit 145.

In general, components of the therapy system 100 may be coupled directly or indirectly. For example, the negative pressure source 105 may be directly coupled to the container 115 and may be indirectly coupled to the dressing 110 through the container 115. Coupling may include fluid, mechanical, thermal, electrical, or chemical coupling (such as a chemical bond), or some combination of coupling in some contexts. For example, the negative pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between-50 mm Hg (−6.7 kPa) and −300 mm Hg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the therapy system 100, such as the negative pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative pressure source 105, the pressure generated by the negative pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, may be any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative pressure source 105, such as a voltage or current, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across the tissue interface 120 under pressure. For example, a manifold may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed, or a secondary fluid path may be provided to facilitate delivering fluid across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 120 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 120 may also vary according to needs of a prescribed therapy. The 25% compression load deflection of the tissue interface 120 may be at least 0.35 pounds per square inch, and the 65% compression load deflection may be at least 0.43 pounds per square inch. In some embodiments, the tensile strength of the tissue interface 120 may be at least 10 pounds per square inch. The tissue interface 120 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 120 may be reticulated polyurethane foam such as found in GRANUFOAM™ dressing or V.A.C. VERAFLO™ dressing, both available from 3M Company.

The thickness of the tissue interface 120 may also vary according to needs of a prescribed therapy. For example, the thickness of the tissue interface may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

The tissue interface 120 may be either hydrophobic or hydrophilic. In an example in which the tissue interface 120 may be hydrophilic, the tissue interface 120 may also wick fluid away from a tissue site, while continuing to distribute negative pressure to the tissue site. The wicking properties of the tissue interface 120 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from 3M Company. Other hydrophilic foams may include those made from polyether. Other foams that may exhibit hydrophilic characteristics include hydrophobic foams that have been treated or coated to provide hydrophilicity.

In some embodiments, the tissue interface 120 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 120 may further serve as a scaffold for new cell-growth, or a scaffold material may be used in conjunction with the tissue interface 120 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38 °C and 10% relative humidity (RH). In some embodiments, an MVTR up to 5,000 grams per square meter per twenty-four hours may provide effective breathability and mechanical properties.

In some example embodiments, the cover 125 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, polyurethane (PU) drape, commercially available from Avery Dennison Corporation, Pasadena, California; polyether block polyamide copolymer (PEBAX), for example, from Arkema S.A., Colombes, France; and INSPIRE® 2301 and INSPIRE® 2327 polyurethane films, commercially available from Exopack Advanced Coatings, Wrexham, United Kingdom. In some embodiments, the cover 125 may comprise INSPIRER 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative pressure source 105 can reduce pressure in the sealed therapeutic environment.

The process of reducing pressure may be described illustratively herein as “delivering,” “distributing,” or “generating” negative pressure, for example. In general, exudate and other fluid flow toward lower pressure along a fluid path. Thus, the term “downstream” typically implies a location in a fluid path relatively closer to a source of negative pressure or further away from a source of positive pressure. Conversely, the term “upstream” implies a location further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source.

Negative pressure applied to the tissue site through the tissue interface 120 in the sealed therapeutic environment can induce macro-strain and micro-strain in the tissue site. Negative pressure can also remove exudate and other fluid from a tissue site, which can be collected in container 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, the controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative pressure source 105 in one or more control modes based on the target pressure and may receive feedback from one or more sensors to maintain the target pressure at the tissue interface 120.

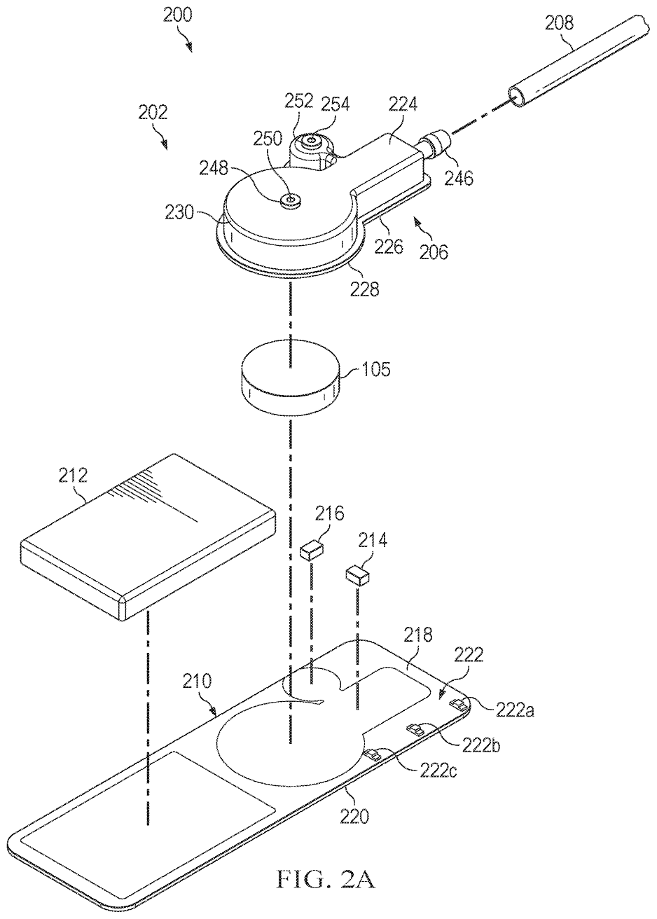

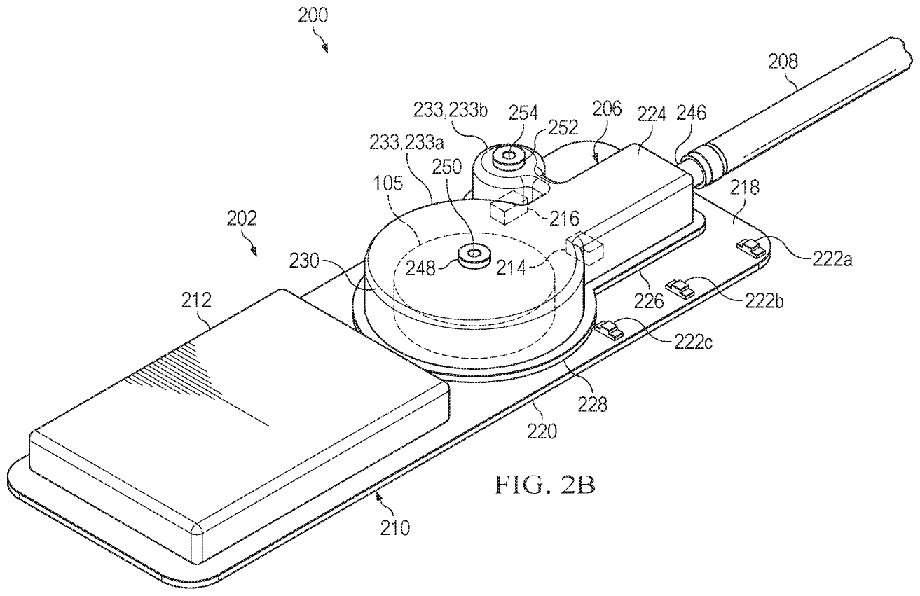

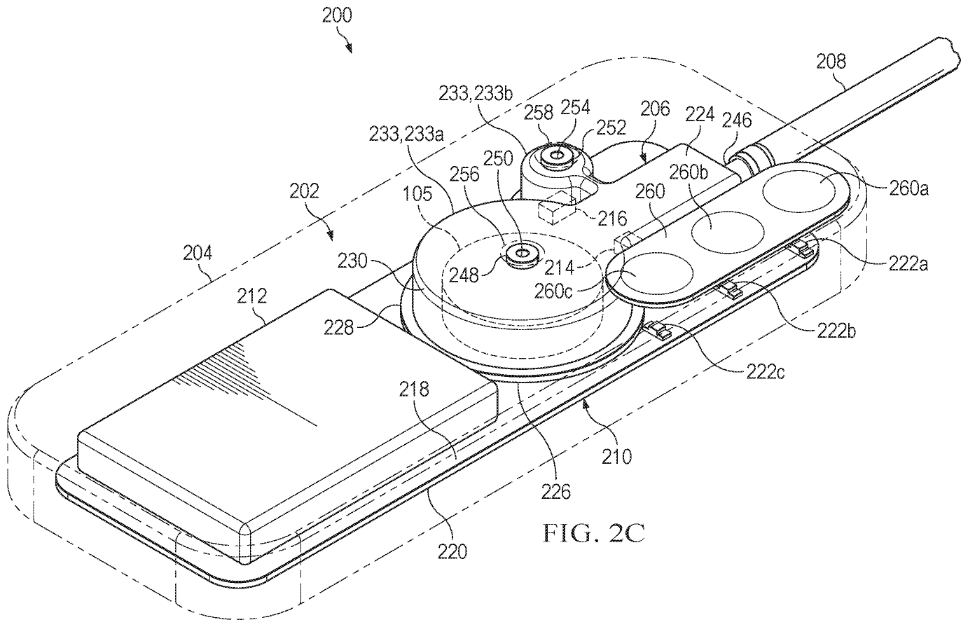

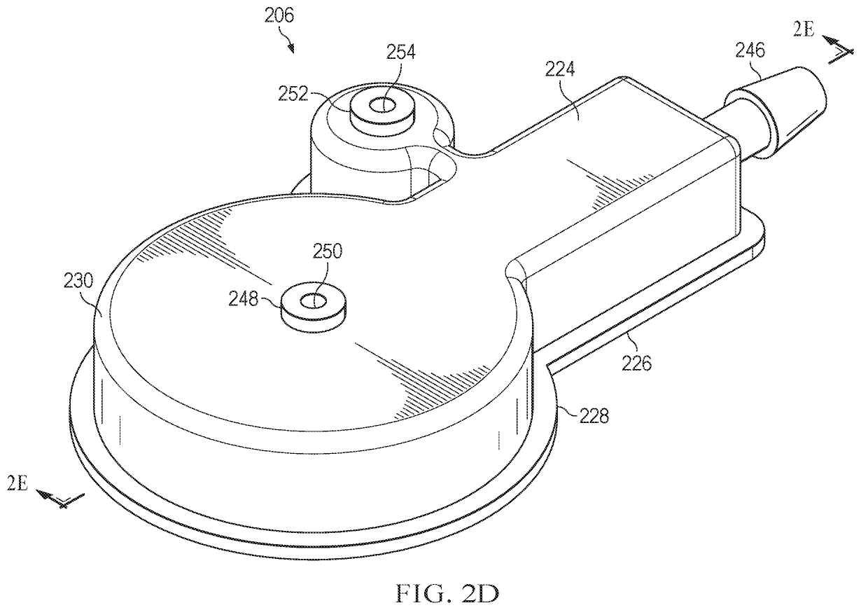

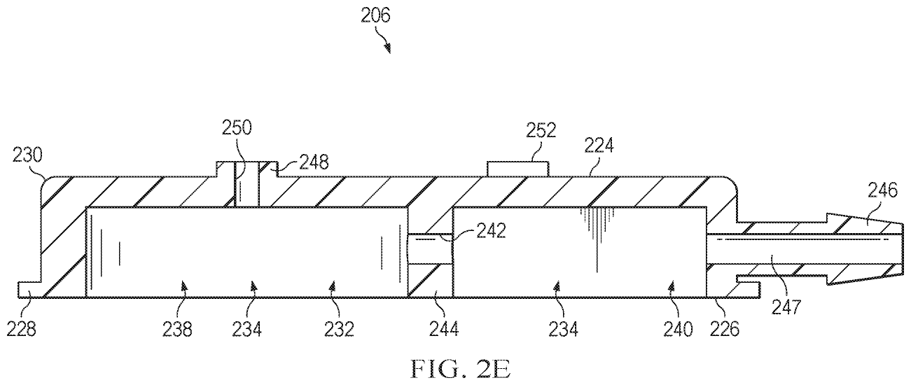

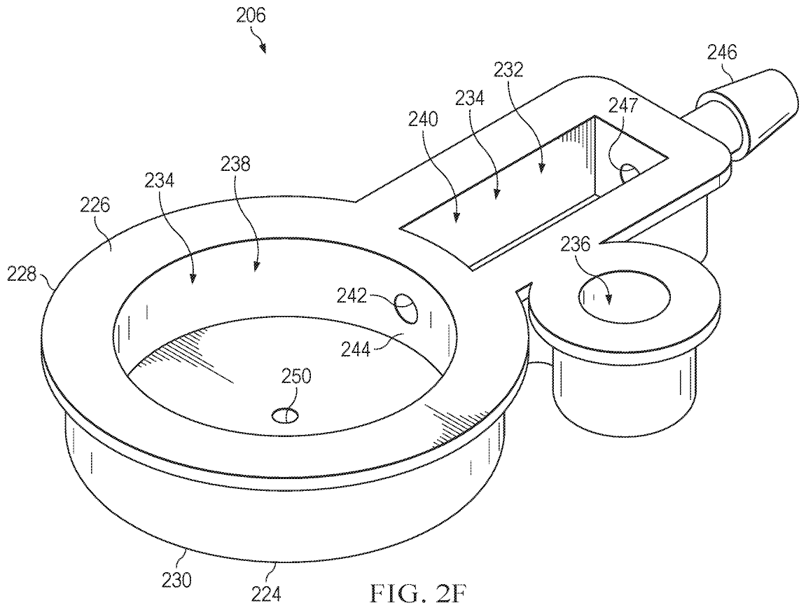

FIGS. 2A-2F show example embodiments of portions of a system 200 for treating a tissue site with negative pressure that may be associated with the therapy system 100. FIG. 2A is an exploded view of a negative pressure device 202 of the system 200. FIG. 2B is a perspective view of the negative pressure device 202. FIG. 2C is a perspective view of the system 200 including the negative pressure device 202 surrounded by an encapsulant 204. FIG. 2D is a perspective view of a device cover or a cover 206 of the negative pressure device 202. FIG. 2E is a cross-sectional view of the cover 206 taken along the line 2E-2E. FIG. 2F is a perspective view of a bottom surface of the cover 206.

The system 200 may be configured to deliver negative pressure to a tissue site and may include the negative pressure device 202 and a tube 208 that may be configured to couple the negative pressure device 202 to the dressing 110, shown in FIG. 1. The negative pressure device 202 may include the cover 206, the negative pressure source 105, the encapsulant 204, and an electric circuit 210 including a power source 212 and at least one sensor, such as a negative pressure sensor 214, and an ambient pressure sensor 216. The negative pressure source 105, the power source 212, the negative pressure sensor 214, and the ambient pressure sensor 216 may each couple with the electric circuit 210. More specifically, each of the negative pressure source 105, the power source 212, the negative pressure sensor 214, and the ambient pressure sensor 216 may be coupled to a first surface 218 of the electric circuit 210. The first surface 218 of the electric circuit 210 may be opposite a second surface 220 that may be a bottom or resting surface such that the negative pressure device 202 can remain in a fixed position while coupled to the dressing 110, in some examples.

The electric circuit 210 may be configured to communicatively couple the negative pressure source 105, the power source 212, the negative pressure sensor 214, and the ambient pressure sensor 216. In some embodiments, the electric circuit 210 may be a printed circuit board. The electric circuit 210 may further include one or more indicators 222. In some embodiments, there may be a first indicator 222a, a second indicator 222b, and a third indicator 222c. The one or more indicators 222 may be configured to indicate a status of the therapy system 100. For example, the one or more indicators 222 may be configured to indicate when there is a blockage in the therapy system 100, when there is a leak in the therapy system 100, when the negative pressure source 105 is operating, and/or other operating conditions of the therapy system 100. In some embodiments, the one or more indicators 222 may be light emitting diodes (LEDs).

The cover 206 may have a top surface 224 and a bottom surface 226 opposite the top surface 224. The bottom surface 226 may include a coupling edge 228 that may extend past a perimeter 230 of the top surface 224. When the negative pressure device 202 is assembled, the cover 206 may be configured to couple to the first surface 218 of the electric circuit 210. More specifically, the bottom surface 226 may be configured to couple to the first surface 218 of the electric circuit 210. When the cover 206 is coupled to the electric circuit, the negative pressure source 105, the negative pressure sensor 214, and the ambient pressure sensor 216 may be disposed between the cover 206 and the first surface 218 of the electric circuit 210 such that the negative pressure source 105, the negative pressure sensor 214, and the ambient pressure sensor 216 are disposed in a cavity 232 of the cover 206. The cavity 232 may be configured to create one or more sealed enclosures 233 around the negative pressure source 105, the negative pressure sensor 214, and the ambient pressure sensor 216.

In some embodiments, the cavity 232 may be divided into more than one cavity. For example, the cavity 232 may include a negative pressure cavity 234 configured to form a first sealed enclosure 233a and a sensing cavity 236 configured to form a second sealed enclosure 233b. The negative pressure cavity 234 may be fluidly isolated from the sensing cavity 236. The negative pressure cavity 234 may be configured to house the negative pressure source 105 and the negative pressure sensor 214. In some embodiments, the negative pressure cavity 234 may have a first section 238 and a second section 240. The first section 238 may be configured to house the negative pressure source 105 and the second section 240 may be configured to house the negative pressure sensor 214. In some embodiments, the first section 238 may be fluidly coupled to the second section 240 through an opening 242 in an interior wall 244 of the cover 206.

In some embodiments, the cover 206 may further include a negative pressure port 246. The negative pressure port 246 may be in fluid communication with the negative pressure cavity 234 through a conduit 247 of the negative pressure port 246 such that the negative pressure cavity 234 is configured to be in fluid communication with the dressing 110. More specifically, the negative pressure port 246 may be fluidly coupled to the second section 240 of the negative pressure cavity 234. In some embodiments, the tube 208 may be configured to couple to the negative pressure port 246 to provide a fluid path between the negative pressure cavity 234 and the dressing 110. The tube 208 may be configured to communicate negative pressure between the negative pressure source 105 and the dressing 110 through the negative pressure cavity 234 and the negative pressure port 246 of the cover 206.

In some embodiments, the cover 206 may further include a first extension 248 with a first exhaust aperture 250 and a second extension 252 with a first ambient pressure sensor aperture 254. The first extension 248 and the second extension 252 may extend from the top surface 224 of the cover 206 away from the bottom surface 226 of the cover 206. The first exhaust aperture 250 may be configured to provide a path for exhaust from the negative pressure source 105 to escape through the cover 206. The first ambient pressure sensor aperture 254 may provide a path from the ambient pressure sensor 216 to an ambient environment surrounding the cover 206.

In some embodiments, not pictured herein, the cover 206 may include more than one element. For example, a portion of the cover 206 defining the negative pressure cavity 234 may be distinct or a separate element from a portion of the cover 206 defining the sensing cavity 236.

In some embodiments, the encapsulant 204 may be configured to surround the cover 206 and at least a portion of the electric circuit 210. As shown in FIG. 2C, the encapsulant 204 is configured to completely surround the cover 206 and the electric circuit 210. The encapsulant 204 may provide protection for the electric circuit 210 from the ambient environment. For example, the encapsulant 204 may protect the electric circuit 210 from dust, liquid, or other potential contaminants. The encapsulant 204 may also protect the electric circuit 210 from mechanical stresses such as dropping the electric circuit 210.

The encapsulant 204 may include a second exhaust aperture 256 that may be substantially aligned with the first exhaust aperture 250. The first exhaust aperture 250 and the second exhaust aperture 256 may be configured to provide a path for exhaust from the negative pressure source 105 to escape from the cover 206 and the encapsulant 204 to an ambient environment surrounding the system 200. In some embodiments, the first extension 248 may couple to the encapsulant 204 to provide a sealed path from the negative pressure cavity 234 to the ambient environment surrounding the system 200. The encapsulant 204 may further include a second ambient pressure sensor aperture 258 that may be substantially aligned with the first ambient pressure sensor aperture 254. The first ambient pressure sensor aperture 254 and the second ambient pressure sensor aperture 258 may be configured to provide a path from the ambient pressure sensor 216 to the ambient environment surrounding the system 200. In some embodiments, the second extension 252 may couple to the encapsulant 204 so provide a sealed path from the sensing cavity 236 to the ambient environment surrounding the system 200.

In some embodiments, the encapsulant 204 may further include an indicator cover 260. The indicator cover 260 may be configured to align with the one or more indicators 222 of the electric circuit 210. The indicator cover 260 may be configured to display a signal generated by the one or more indicators 222 such that the signal is easily observed by a user. In some embodiments, the indicator cover 260 may have a first section 260a configured to align with the first indicator 222a, a second section 260b configured to align with the second indicator 222b, and a third section 260c configured to align with the third indicator 222c.

In some embodiments, not pictured herein, the encapsulant 204 may be applied to the negative pressure device 202 without the use of the cover 206. For example, the encapsulant 204 may be configured to create a sealed enclosure around at least the negative pressure source 105 and the negative pressure sensor 214 such that the negative pressure source 105 and the negative pressure sensor 214 may be fluidly coupled to the dressing 110 through the tube 208 but may be isolated from the remainder of the negative pressure device 202.

In some embodiments, the electric circuit 210 may be a rigid printed circuit board, a flexible printed circuit board, or another type of circuit that provides connections between the electric components of the negative pressure device 202. The cover 206 may be either rigid or flexible such that it is able to couple to the electric circuit 210 to create a sealed enclosure around the necessary electric components of the negative pressure device 202. In some embodiments, the cover 206 may be formed from plastic.

The encapsulant 204 may be either rigid or flexible. In some embodiments, the encapsulant 204 may be UV cured to achieve its final shape and rigidity. If the encapsulant 204 is flexible, it may be formed from a silicone encapsulant material that may be UV curable. In some embodiments when the encapsulant 204 is flexible, the encapsulant may be transparent to allow the cover 206, the electric circuit 210, and the other components of the negative pressure device 202 to be visible through the encapsulant. The silicone encapsulant material may be elastomeric and may not generate heat while it is formed around the electric circuit 210 and the cover 206 which may help to reduce any risk of damaging any of the components that are coupled to the electric circuit 210. The silicone encapsulant material may be used as the encapsulant 204 when the electric circuit 210 is a flexible printed circuit board. In embodiments where the encapsulant 204 is rigid, the encapsulant 204 may be formed from an epoxy potting compound. When using an epoxy potting compound, heat can be generated during the potting process when a hardener of the epoxy potting compound is mixed with a base resin of the epoxy potting compound. The epoxy potting compound may produce a dark, opaque appearance for the encapsulant 204. In embodiments where the encapsulant 204 is rigid, the encapsulant 204 may be wrapped around the other components of the negative pressure device 202 to add surface finish and to emphasize details of the negative pressure device 202. In some embodiments, the encapsulant 204 may be formed from urethane and acrylic compounds. In some embodiments, the use of a rigid printed circuit board and a soft, flexible encapsulant may improve robustness of the system 200.

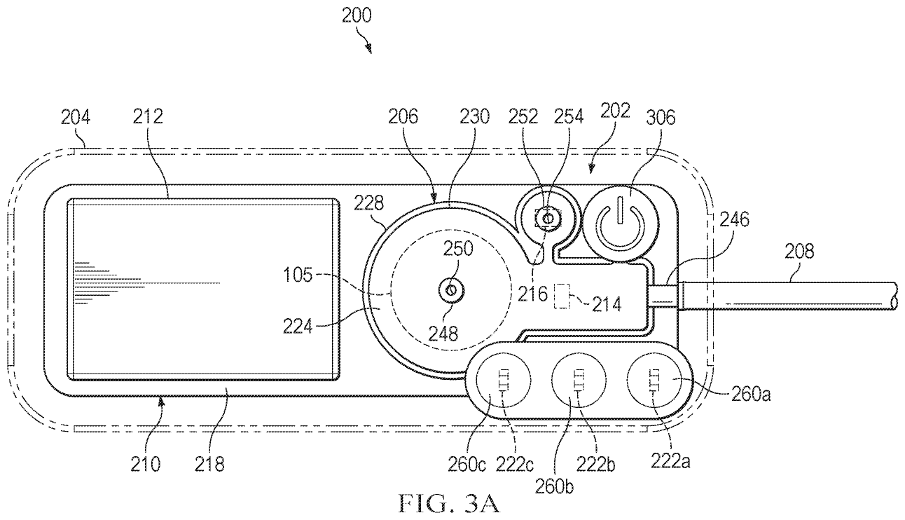

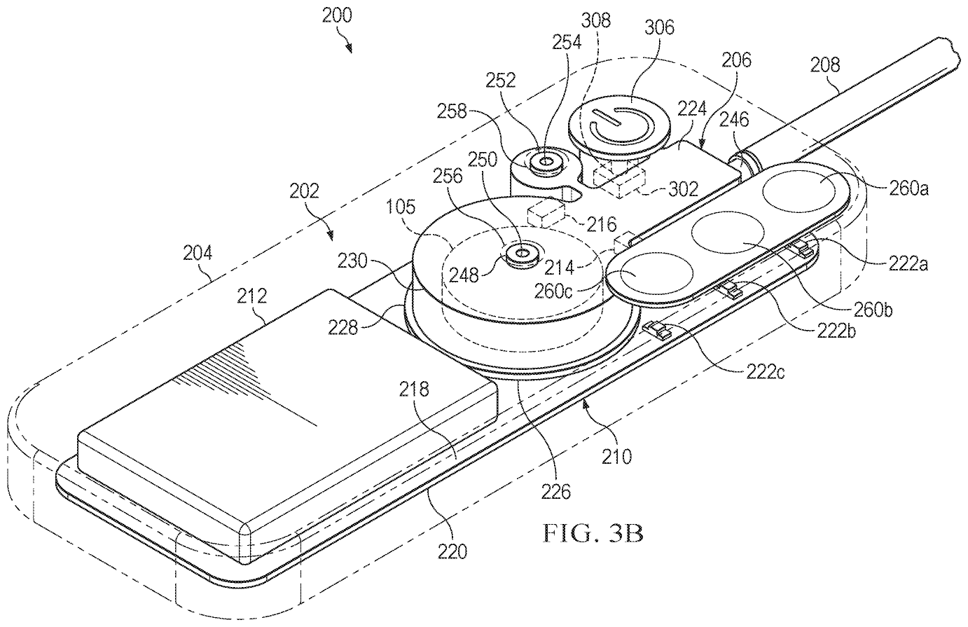

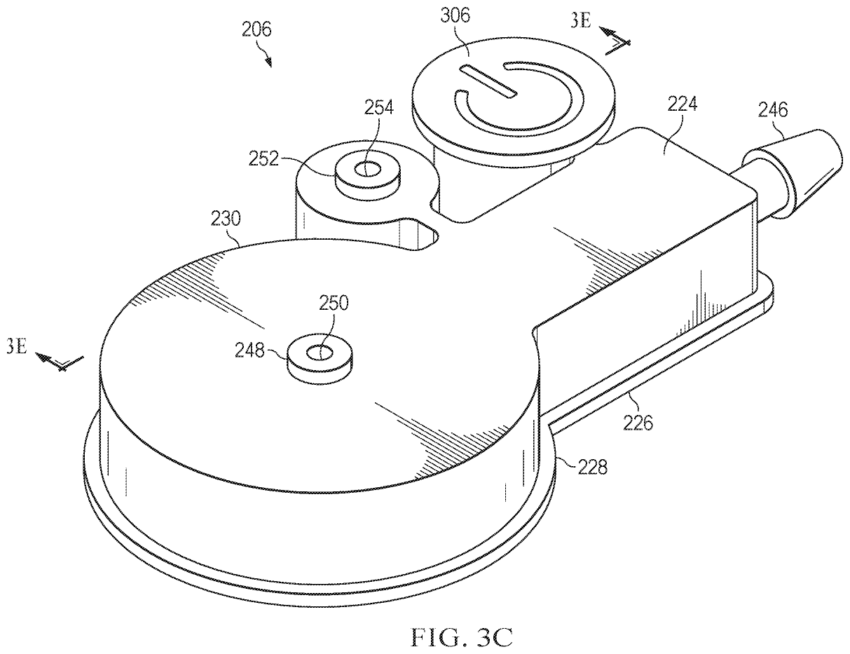

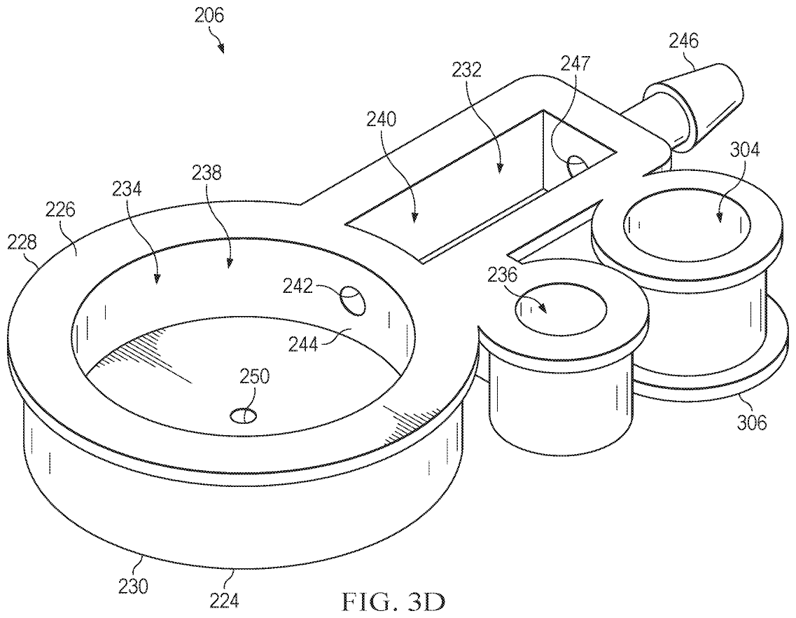

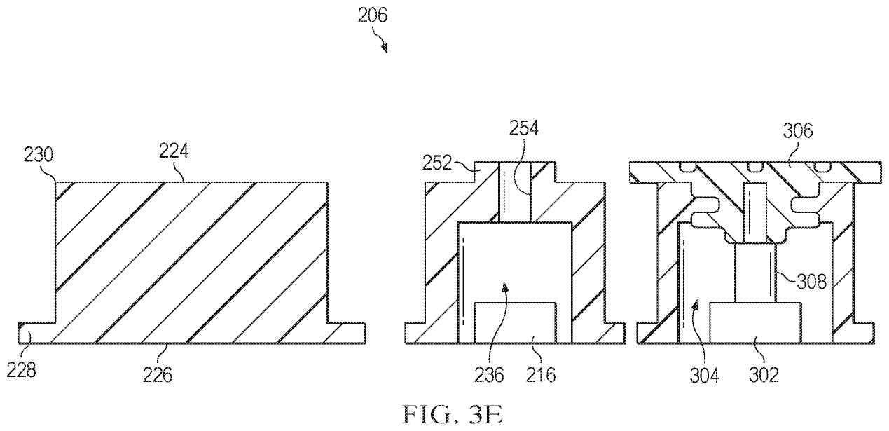

Referring to FIGS. 3A to 3E, another embodiment of the system 200 is illustrated. FIG. 3A shows a top view of the system 200 with the negative pressure device 202 surrounded by the encapsulant 204. FIG. 3B shows a perspective view of the system 200 of FIG. 3A. FIG. 3C is a perspective view of the cover 206 of the negative pressure device 202 of FIG. 3A. FIG. 3D is a bottom perspective view of the cover 206 of FIG. 3C. FIG. 3E is a cross-sectional view of the cover 206 of FIG. 3C taken along line 3E-3E.

The system 200 may be substantially as described above with reference to FIGS. 2A-2F. The negative pressure device 202 may include the electric circuit 210, the negative pressure source 105, the power source 212, the negative pressure sensor 214, the ambient pressure sensor 216, and the cover 206. The negative pressure device 202 of FIGS. 3A-3E may additionally include a switch 302. The switch 302 may be coupled to the first surface 218 of the electric circuit 210 similar to the negative pressure source 105, the negative pressure sensor 214, the ambient pressure sensor 216, and the power source 212. The switch 302 may be configured to actuate at least one element of the negative pressure device 202. For example, the switch 302 may be configured to actuate the negative pressure source 105. When the negative pressure source 105 is actuated, the dressing 110 may be drawn down to a desired negative pressure. The switch 302 may also be configured to power off the negative pressure source 105 such that negative pressure is no longer being applied to the dressing 110.

In some embodiments, the cavity 232 of the cover 206 may further include a switch cavity 304. The switch cavity 304 may be fluidly isolated from the negative pressure cavity 234 and the sensing cavity 236 and may be configured to house the switch 302. The cover 206 may further include a button overmount 306. The button overmount 306 may be coupled to the top surface 224 of the cover 206 and may be configured to engage an extension 308 of the switch 302. The extension 308 of the switch 302 may be configured to engage the switch 302 when a force is applied to the button overmount 306. In some embodiments, the button overmount 306 may be formed from a material that is more flexible than the material that the cover 206 is formed from. When the button overmount 306 is more flexible than the remainder of the cover 206, the force necessary to actuate the switch 302 may be easily achieved by pressing on the encapsulant 204 adjacent to the button overmount 306.

In some embodiments, the cover 206 may include more than one element. For example, a portion of the cover 206 defining the switch cavity 304 may be distinct from a portion of the cover 206 defining the negative pressure cavity 234 and the sensing cavity 236. In embodiments where the portion of the cover 206 defining the switch cavity 304 is distinct from the portion of the cover 206 defining the negative pressure cavity 234 and the sensing cavity 236, the portion of the cover 206 defining the switch cavity 304 may be manufactured to have a different rigidity than the portion of the cover 206 defining the negative pressure cavity 234 and the sensing cavity 236.

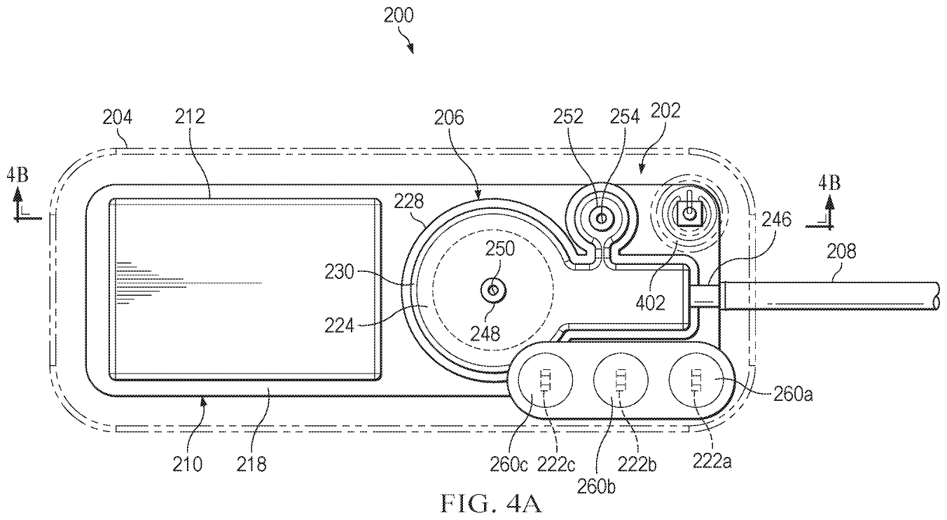

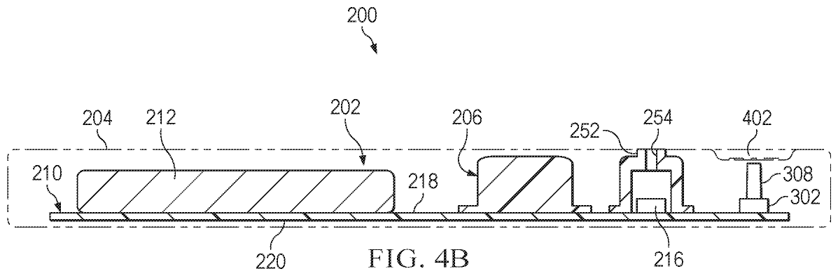

Referring to FIGS. 4A and 4B, another embodiment of the system 200 is shown. FIG. 4A shows a top view of the system 200 with the negative pressure device 202 surrounded by the encapsulant 204, and FIG. 4B shows a cross-sectional view of the system 200 of FIG. 4A taken along line 4B-4B.

The system 200 of FIGS. 4A and 4B may be similar to the system 200 of FIGS. 3A-3E. The negative pressure device 202 may include the electric circuit 210, the negative pressure source 105, the power source 212, the negative pressure sensor 214, the ambient pressure sensor 216, the cover 206, and the switch 302. The cover 206 of FIGS. 4A and 4B may be substantially similar to the cover 206 of FIGS. 2A-2F. Thus, the switch 302 may not be enclosed by the cover 206.

In some embodiments, the encapsulant 204 may have a button 402 formed into the encapsulant 204. The extension 308 of the switch 302 may extend from the switch 302 proximate to the button 402 of the encapsulant 204 such that the switch may be engaged by a force being applied to the button 402 of the encapsulant 204. The encapsulant 204 may be formed from any of the materials described above that enable a force applied to the encapsulant 204 to flex the button 402 enough to engage the extension 308 to actuate the switch 302.

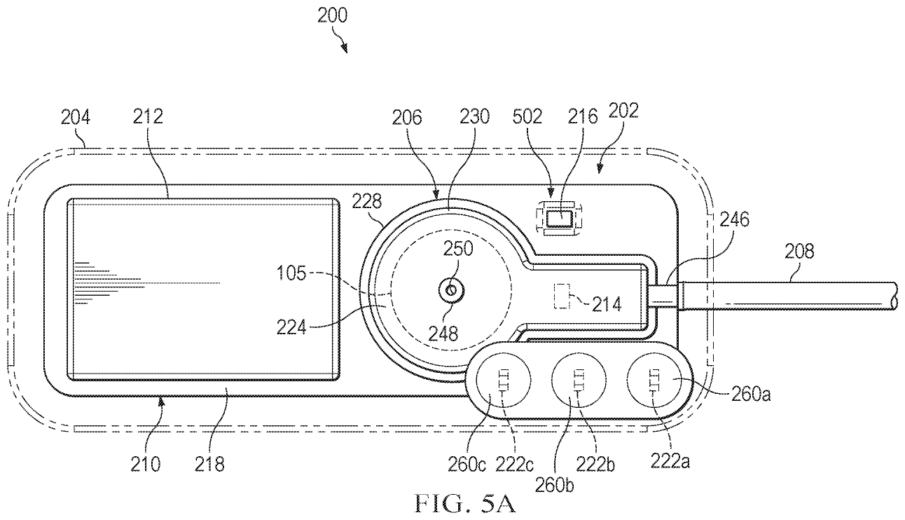

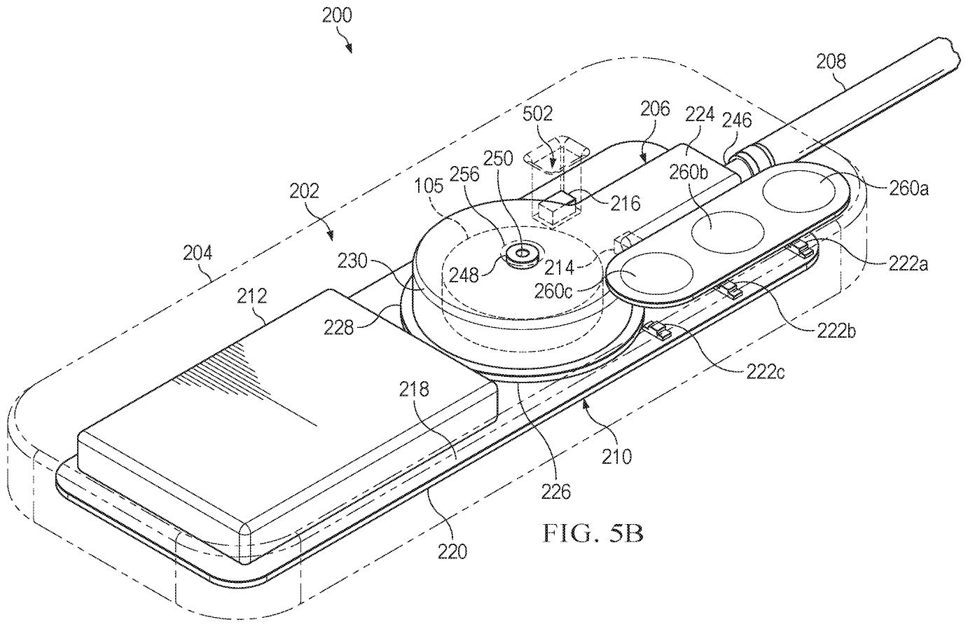

Referring to FIGS. 5A and 5B, another embodiment of the system 200 is shown. FIG. 5A shows a top view of the system 200 with the negative pressure device 202 surrounded by the encapsulant 204, and FIG. 5B shows a perspective view of the system 200 of FIG. 5A.

The system 200 of FIGS. 5A and 5B may be similar to the system 200 of FIGS. 2A-2F. The negative pressure device 202 may include the electric circuit 210, the negative pressure source 105, the power source 212, the negative pressure sensor 214, the ambient pressure sensor 216, and the cover 206. The cover 206 of FIGS. 5A and 5B may include the negative pressure cavity 234 but does not include the sensing cavity 236. More specifically, the ambient pressure sensor 216 may not be enclosed by the cover 206.

The encapsulant 204 may include an opening 502 configured to expose the ambient pressure sensor 216 to the ambient environment surrounding the encapsulant 204. In some embodiments, the encapsulant 204 may couple to the electric circuit 210 surrounding the ambient pressure sensor 216 such that the ambient environment surrounding the encapsulant 204 does not contact any portion of the negative pressure device 202 through the opening 502 other than the ambient pressure sensor 216.

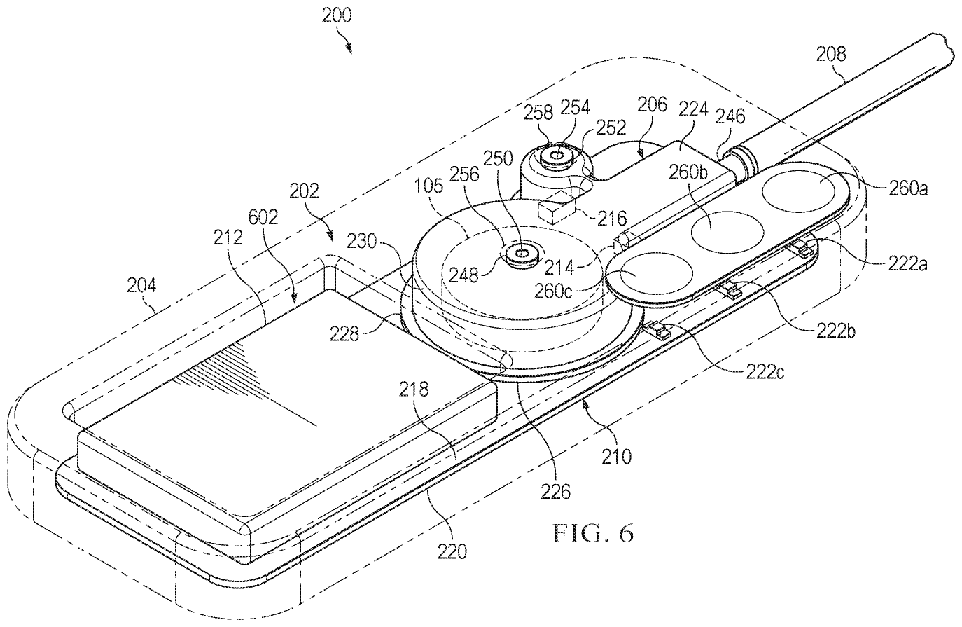

Referring to FIG. 6, another embodiment of the system 200 is shown. FIG. 6 shows a perspective view of the system 200 with the negative pressure device 202 surrounded by the encapsulant 204.

The system 200 of FIG. 6 may be similar to the system 200 of FIGS. 2A-2F. The negative pressure device 202 may include the electric circuit 210, the negative pressure source 105, the power source 212, the negative pressure sensor 214, the ambient pressure sensor 216, and the cover 206. The encapsulant 204 may include an opening 602 configured to expose the power source 212 to the ambient environment surrounding the encapsulant 204. In some embodiments, the encapsulant 204 may couple to the electric circuit 210 surrounding the power source 212 such that the ambient environment surrounding the system 200 does not contact any portion of the negative pressure device 202 through the opening 602 other than the power source 212. When the power source 212 is exposed to the ambient environment surrounding the encapsulant 204, the power source 212 may be charged with an external charger or may be replaced with another power source without disturbing remainder of the negative pressure device 202 or the encapsulant 204.

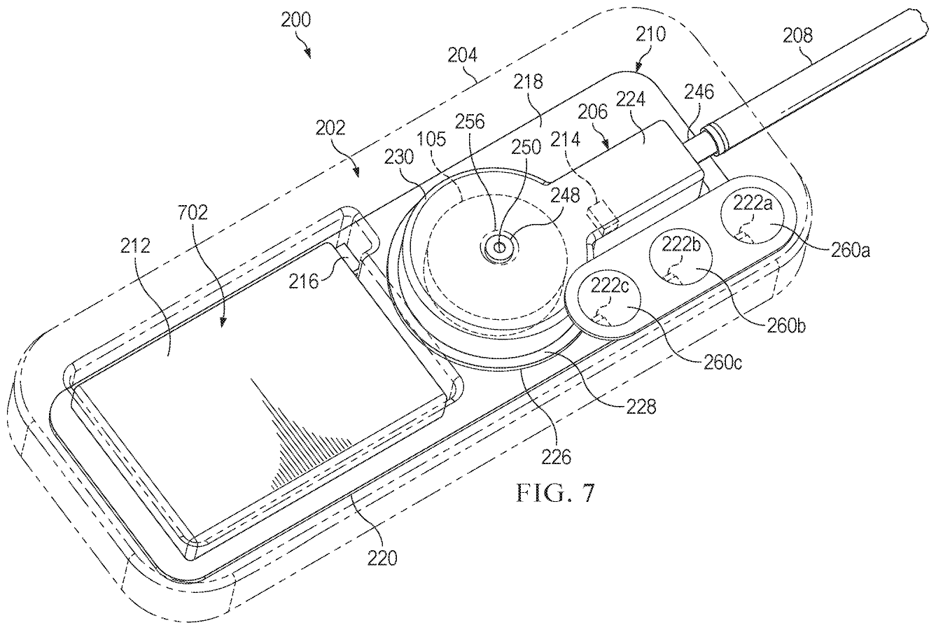

Referring to FIG. 7, another embodiment of the system 200 is shown. FIG. 7 shows a perspective view of the system 200 with the negative pressure device 202 surrounded by the encapsulant 204.

The system 200 of FIG. 7 may be similar to the system 200 of FIGS. 2A-2F. The negative pressure device 202 may include the electric circuit 210, the negative pressure source 105, the power source 212, the negative pressure sensor 214, the ambient pressure sensor 216, and the cover 206. In some embodiments, the ambient pressure sensor 216 may be disposed on the first surface 218 of the electric circuit proximate to the power source 212. When the ambient pressure sensor 216 is disposed proximate to the power source 212, the ambient pressure sensor 216 may not be enclosed by the cover 206. Thus, the cover 206 may include the negative pressure cavity 234 but does not include the sensing cavity 236.

The encapsulant 204 may include an opening 702 configured to expose the power source 212 and the ambient pressure sensor 216 to the ambient environment surrounding the encapsulant 204. In some embodiments, the encapsulant 204 may couple to the electric circuit 210 surrounding the power source 212 and the ambient pressure sensor 216 such that the ambient environment surrounding the system 200 does not contact any portion of the negative pressure device 202 through the opening 702 other than the power source 212 and the ambient pressure sensor 216. When the power source 212 is exposed to the ambient environment surrounding the encapsulant 204, the power source 212 may be charged with an external charger or may be replaced with another power source without disturbing remainder of the negative pressure device 202 or the encapsulant 204. The ambient pressure sensor 216 may be able to sense an ambient pressure surrounding the encapsulant 204 directly through the opening 702.

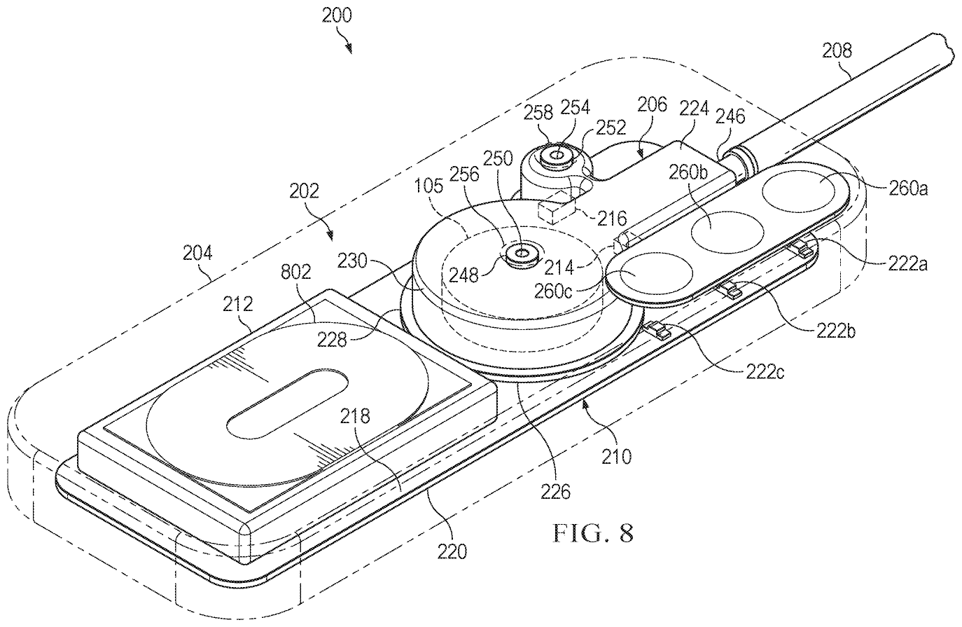

Referring to FIG. 8, another embodiment of the system 200 is shown. FIG. 8 shows a perspective view of the system 200 with the negative pressure device 202 surrounded by the encapsulant 204.

The system 200 of FIG. 8 may be similar to the system 200 of FIGS. 2A-2F. The negative pressure device 202 may include the electric circuit 210, the negative pressure source 105, the power source 212, the negative pressure sensor 214, the ambient pressure sensor 216, and the cover 206. A wireless charging coil 802 may be coupled to the electric circuit 210. The wireless charging coil 802 may be configured to couple with an external charger to charge the power source 212 through the encapsulant 204. The wireless charging coil 802 may be coupled to a surface of the encapsulant 204 that couples to the power source 212 or to a surface of the encapsulant 204 opposite the power source 212 such that the wireless charging coil 802 can provide a charge to the power source 212.

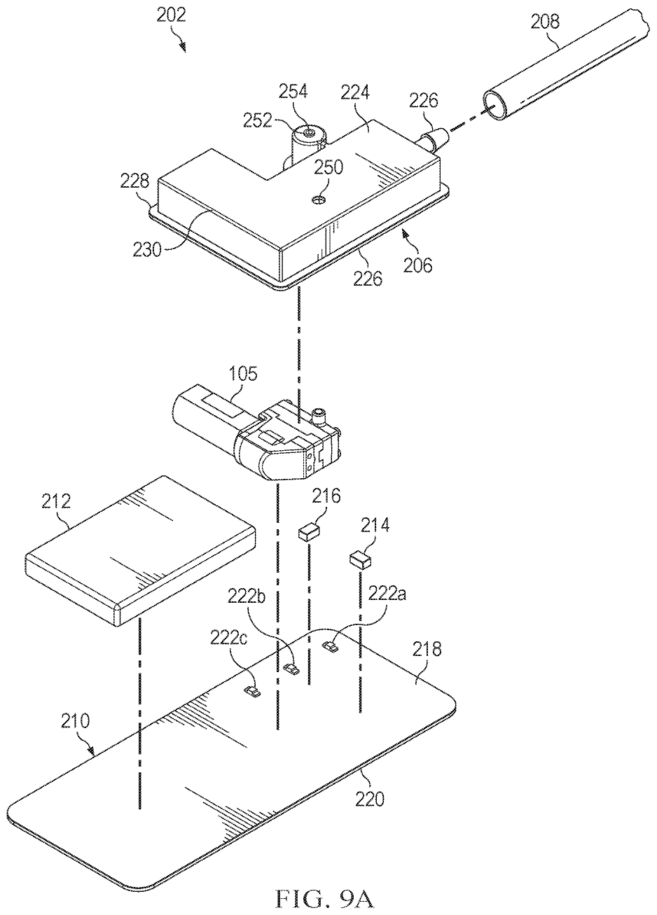

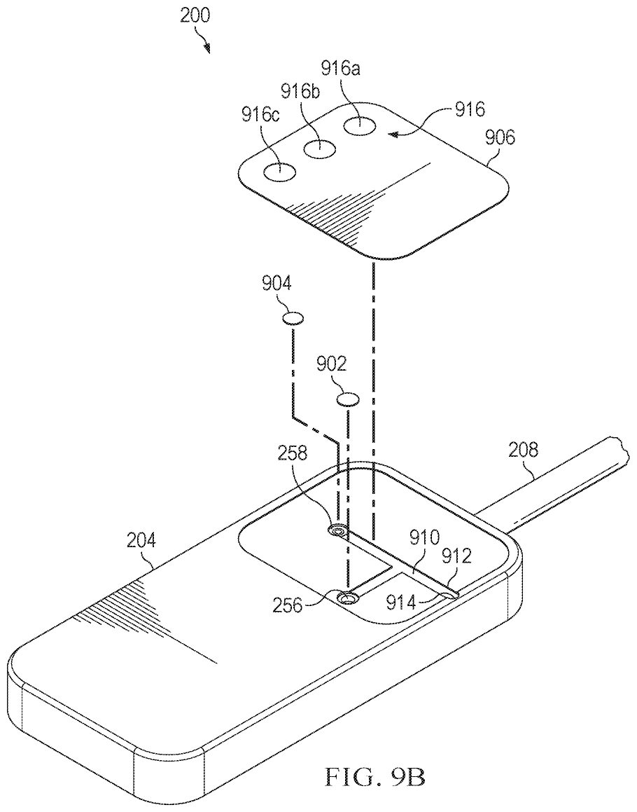

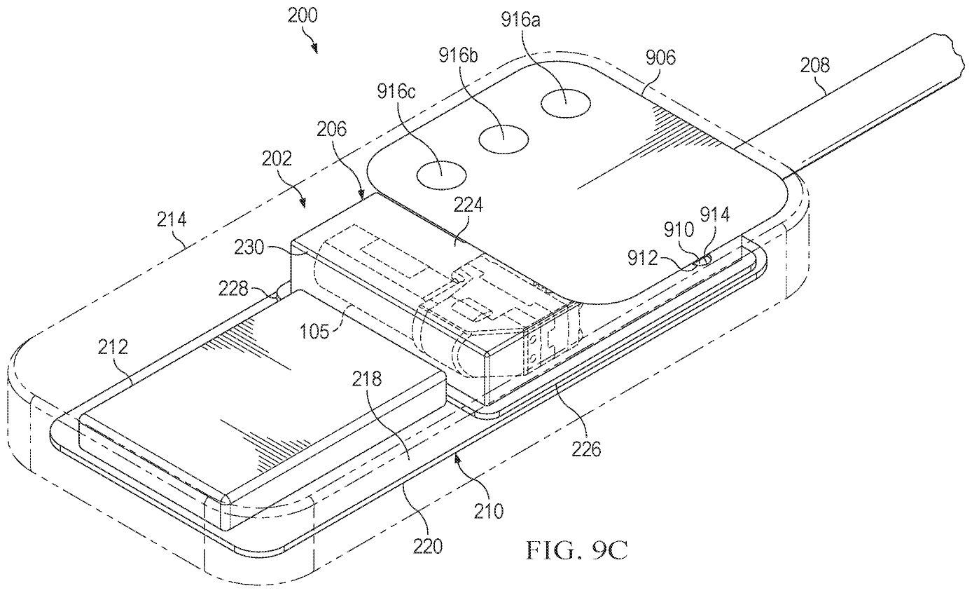

Referring to FIGS. 9A-9C, another embodiment of the system 200 is shown. The negative pressure device 202 may include the electric circuit 210, the power source 212, the negative pressure sensor 214, the ambient pressure sensor 216, the negative pressure source 105, and the cover 206. The negative pressure source 105 may be a diaphragm pump in some embodiments. The cover 206 may be configured to create a sealed enclosure around the negative pressure source 105, the ambient pressure sensor 216, and the negative pressure sensor 214 substantially as described above with reference to FIGS. 2A-2F.

In some embodiments, the system 200 may further include a first filter 902, a second filter 904, and a membrane 906. The first filter 902, the second filter 904, and the membrane 906 may be configured to couple to the encapsulant 204 opposite the negative pressure device 202. The first filter 902 may be configured to couple to the second exhaust aperture 256 of the encapsulant 204 and the second filter 904 may be configured to couple to the second ambient pressure sensor aperture 258 of the encapsulant. The membrane 906 may be configured to secure to the encapsulant 204 to dispose the first filter 902 and the second filter 904 between the membrane 906 and the encapsulant 204.

In some embodiments, the encapsulant 204 may include an ambient environment pathway 910. The ambient environment pathway 910 may provide a path from the first filter 902 and the second filter 904 to the ambient environment surrounding the system 200. In some embodiments, the ambient environment pathway 910 may extend from both the second exhaust aperture 256 and the second ambient pressure sensor aperture 258 to a side 912 of the encapsulant 204. An end 914 of the ambient environment pathway 910 may extend past the membrane 906 when the membrane 906 is coupled to the encapsulant 204.

In some embodiments, the membrane 906 may further include an indicator window 916. The indicator window 916 may include a first window 916a, a second window 916b, and a third window 916c. The first window 916a may be configured to align with the first indicator 222a, the second window 916b may be configured to align with the second indicator 222b, and the third window 916c may be configured to align with the third indicator 222c. In some embodiments, the encapsulant 204 and/or the membrane 906 may be substantially opaque such that the indicator window 916 provides the only visualization of the system 200 through the encapsulant 204 and/or the membrane 906.

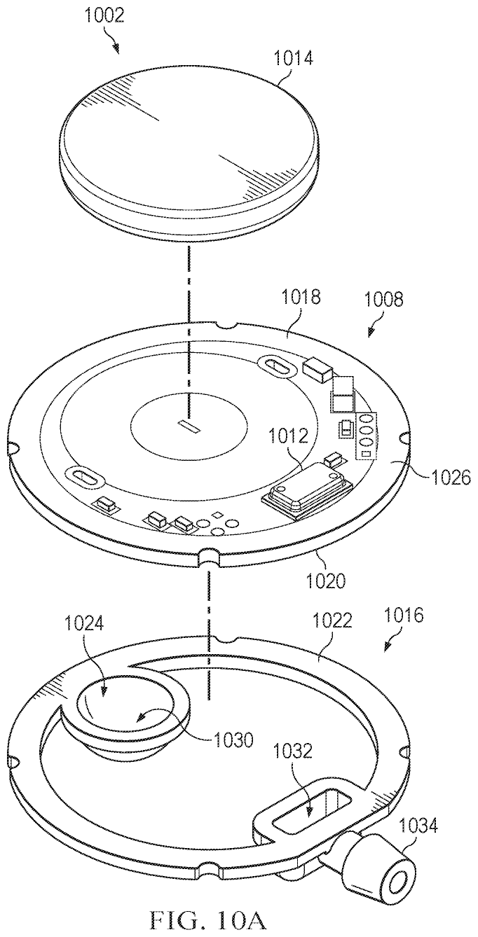

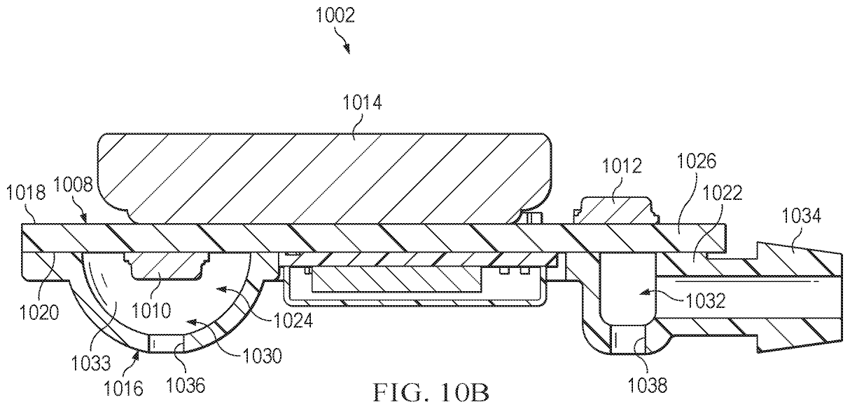

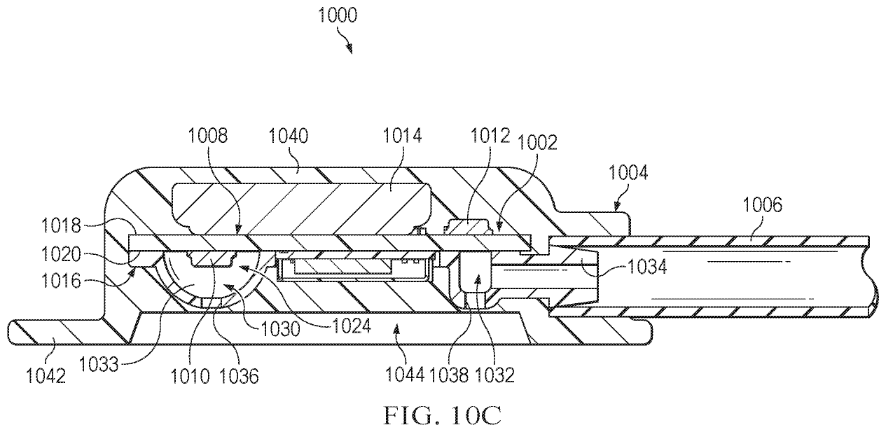

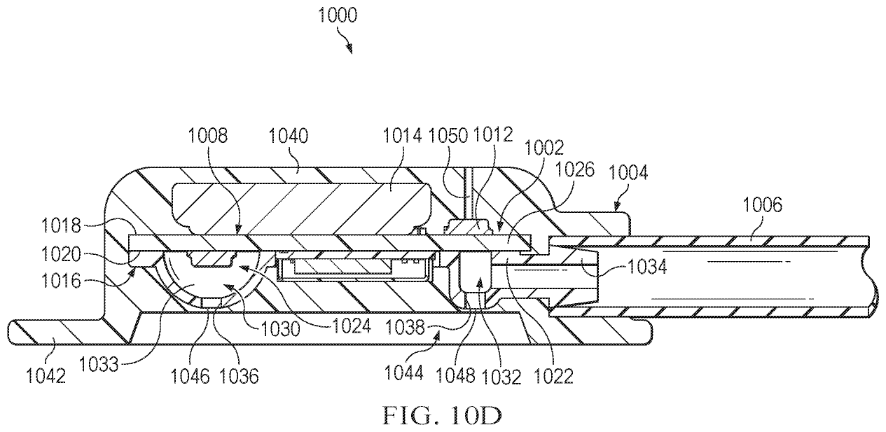

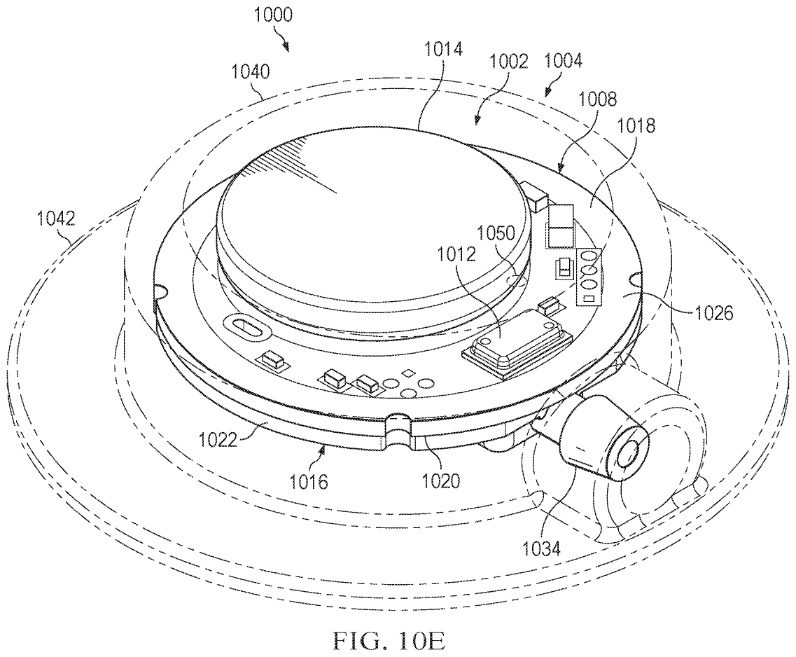

Referring to FIGS. 10A-10E, a system 1000 suitable for sensing properties of a tissue site is shown. FIG. 10A shows an exploded view of a wound sensing device 1002 of the system 1000. FIG. 10B shows a cross-sectional view of the wound sensing device 1002 of FIG. 10A. FIG. 10C shows a cross-sectional view of the system 1000 with the wound sensing device 1002 surrounded by an encapsulant 1004. FIG. 10D shows the system of FIG. 10C with sensing apertures drilled into the encapsulant 1004. FIG. 10E shows a perspective view of the system 1000.

The system 1000 may be configured to be disposed on the dressing 110 opposite the tissue site to be able to sense properties at the tissue site. The system 1000 may include the wound sensing device 1002, the encapsulant 1004, and a tube 1006 that may be configured to couple to the wound sensing device 1002 to fluidly couple the dressing 110 to the negative pressure source 105. The wound sensing device 1002 may include an electric circuit 1008, a negative pressure sensor 1010, an ambient pressure sensor 1012, a power source 1014, and a cover 1016. In some embodiments, the wound sensing device 1002 may not include the ambient pressure sensor 1012. The negative pressure sensor 1010, the ambient pressure sensor 1012, and the power source 1014 may be configured to couple to the electric circuit 1008 such that the electric circuit 1008 is configured to communicatively couple the negative pressure sensor 1010, the ambient pressure sensor 1012, and the power source 1014.

In some embodiments, the electric circuit 1008 may include a first surface 1018 and a second surface 1020 opposite the first surface 1018. The power source 1014 and the ambient pressure sensor 1012 may be configured to couple to the first surface 1018 of the electric circuit 1008, and the negative pressure sensor 1010 may be configured to couple to the second surface 1020 of the electric circuit 1008 in some embodiments.

In some embodiments, the cover 1016 may include a ring 1022 and at least one cavity 1024. The cover 1016 may be configured to couple to the second surface 1020 of the electric circuit 1008. The ring 1022 of the cover 1016 may be configured to couple to a perimeter 1026 of the second surface 1020 of the electric circuit 1008. The at least one cavity 1024 of the cover 1016 may include a sensing cavity 1030 and a negative pressure cavity 1032. The sensing cavity 1030 may be configured to create a sealed enclosure 1033 around the negative pressure sensor 1010. The sensing cavity 1030 and the sealed enclosure 1033 can be fluidly isolated from the negative pressure cavity 1032 and the ambient pressure sensor 1012. The cover 1016 may further include a negative pressure port 1034 that may be in fluid communication with the negative pressure cavity 1032. The negative pressure port 1034 may be configured to couple with the tube 1006 to fluidly couple the negative pressure source 105 to the dressing 110 through the negative pressure cavity 1032 of the cover 1016.

The cover 1016 may further include a sensing aperture 1036 and a negative pressure opening 1038. The sensing aperture 1036 may be an aperture or an opening formed through a portion of the cover 1016 defining the sensing cavity 1030. The sensing aperture 1036 may be configured to expose the sensing cavity 1030 and the negative pressure sensor 1010 to the dressing 110. The negative pressure opening 1038 may be an aperture or an opening formed through a portion of the cover 1016 defining the negative pressure cavity 1032. The negative pressure opening 1038 may be configured to provide a path from the negative pressure cavity 1032 to the dressing 110.

The encapsulant 1004 may be configured to enclose the wound sensing device 1002 except for the negative pressure port 1034 such that the encapsulant is proximate to the tube 1006 and the wound sensing device 1002 when the tube is coupled to the negative pressure port 1034. In some embodiments, the encapsulant 1004 may include a surrounding portion 1040 and a base portion 1042. The surrounding portion 1040 may be configured to surround the wound sensing device 1002. The base portion 1042 may extend from the surrounding portion 1040 and may be a coupling surface for the system 1000 to couple to the dressing 110. In some embodiments, the base portion 1042 may be in the form of a ring such that there is an opening 1044 formed between the system 1000 and the dressing 110 when the system 1000 is disposed on the dressing 110.

In some embodiments, the encapsulant 1004 may include a first opening 1046 and a second opening 1048. The first opening 1046 may be configured to align with the sensing aperture 1036 of the cover 1016 and the second opening 1048 may be configured to align with the negative pressure opening 1038 of the cover 1016. The negative pressure sensor 1010 may be configured to sense a pressure at the dressing 110 through the sensing aperture 1036 and the first opening 1046 and negative pressure may be delivered to the dressing 110 through the negative pressure opening 1038 and the second opening 1048.

In embodiments that include the ambient pressure sensor 1012, the encapsulant 1004 may further include an ambient pressure sensor opening 1050. The ambient pressure sensor opening 1050 may be formed through the encapsulant 1004 to provide fluid communication between the ambient pressure sensor 1012 and an ambient environment surrounding the system 1000.

In some embodiments, the first opening 1046, the second opening 1048, and the ambient pressure sensor opening 1050 may formed through the encapsulant 1004 after the encapsulant 1004 is formed around the wound sensing device 1002. In some embodiments, the first opening 1046, the second opening 1048, and the ambient pressure sensor opening 1050 may formed by drilling through the encapsulant 1004.

In some embodiments, not pictured herein, the encapsulant 1004 may be formed around only a portion of the wound sensing device 1002. The encapsulant 1004 may include a power source opening similar to opening 602 of FIG. 6. The power source opening may enable the power source 1014 to be removed, replaced, or recharged without impacting the remainder of the wound sensing device 1002.

The encapsulant 1004, the electric circuit 1008, and the cover 1016 may be formed from any of the materials described above with reference to FIGS. 2A-2F.

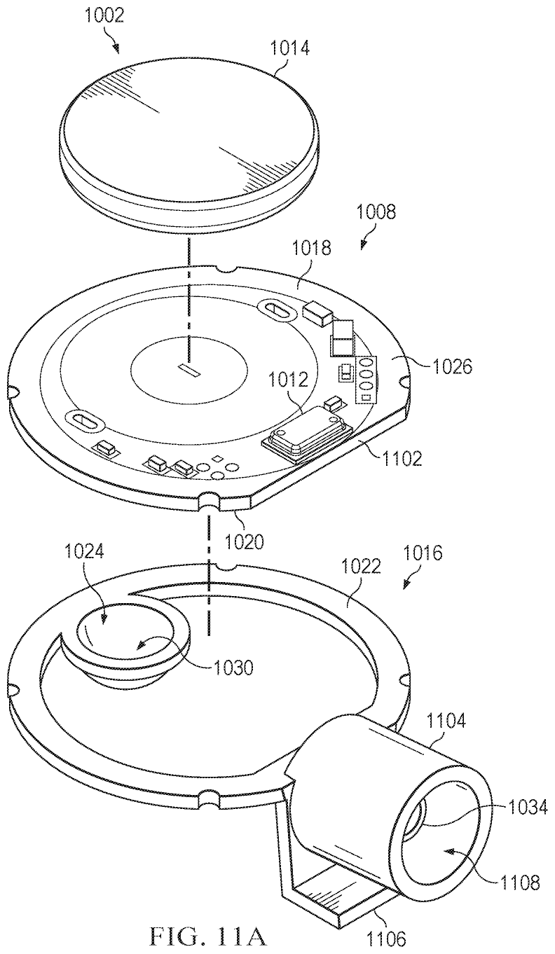

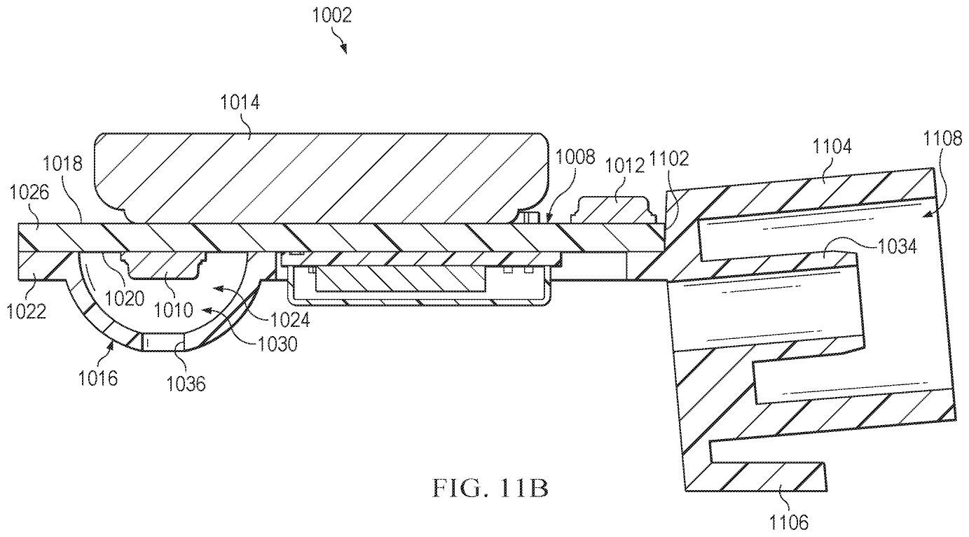

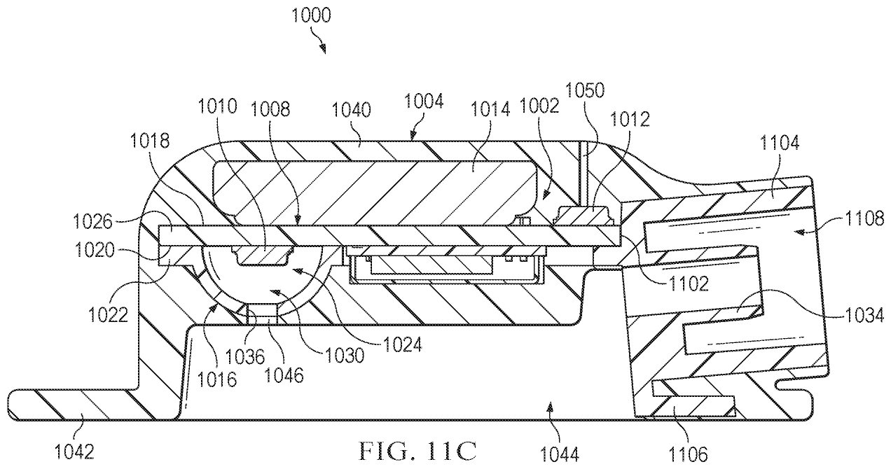

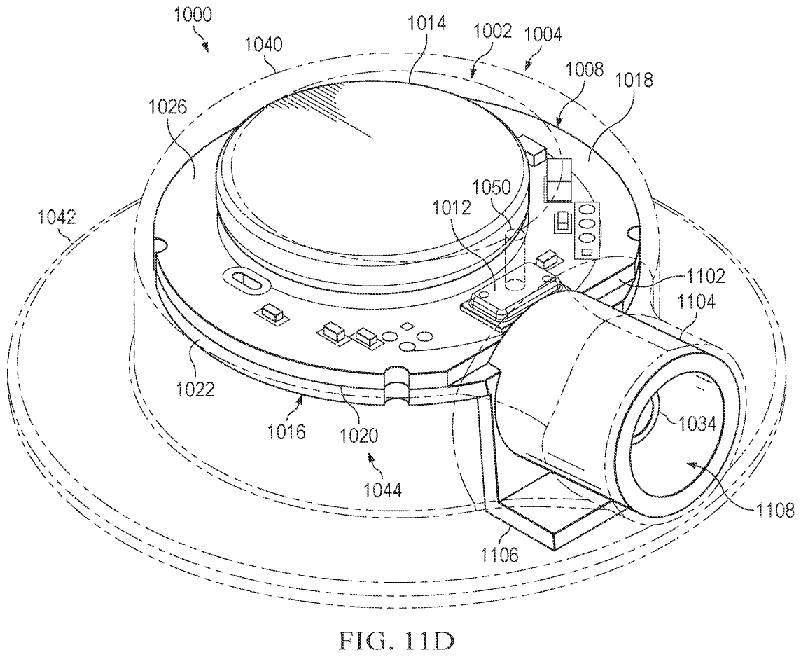

Referring to FIGS. 11A-11D, another embodiment of the system 1000 is shown. FIG. 11A is an exploded view of a wound sensing device 1002 of the system 1000. FIG. 11B is a cross-sectional view of the wound sensing device 1002 of FIG. 11A. FIG. 11C shows a cross-sectional view of the system 1000 with the wound sensing device 1002 surrounded by the encapsulant 1004. FIG. 11D shows a perspective view of the system 1000.

As described above with reference to FIGS. 10A-10E, the system 1000 may include the wound sensing device 1002, the encapsulant 1004, and the tube 1006. The wound sensing device 1002 may include the electric circuit 1008, the negative pressure sensor 1010, the ambient pressure sensor 1012, the power source 1014, and the cover 1016. The electric circuit 1008 may be substantially as described above with reference to FIGS. 10A-10E but may include a coupling edge 1102. The coupling edge 1102 may be configured to couple with a port enclosure 1104 of the cover 1016.

The cover 1016 may include the ring 1022 and the at least one cavity 1024. The at least one cavity may be the sensing cavity 1030. The cover 1016 may further include the negative pressure port 1034 which may be surrounded by the port enclosure 1104. The port enclosure 1104 may be configured to receive the tube 1006 when the tube 1006 is coupled to the negative pressure port 1034 to couple the negative pressure source 105 to the dressing 110. In some embodiments, the cover 1016 may further include an extension 1106 extending from the ring 1022 of the cover 1016 proximate to the port enclosure 1104. The extension 1106 may extend from the ring 1022 opposite the electric circuit 1008. In some embodiments, the extension 1106 may provide structure or support to the wound sensing device 1002.

The encapsulant 1004 may be configured to enclose the wound sensing device 1002 except for an opening 1108 of the port enclosure 1104 that is configured to receive the tube 1006. In some embodiments, the negative pressure port 1034 may open directly into the opening 1044 between the encapsulant 1004 and the dressing 110. Thus, a hole or opening may not need to be formed through the encapsulant 1004 to enable the negative pressure source 105 to expose the dressing 110 to a negative pressure.

Also described herein is a method of manufacturing a device such as the system 200 or the system 1000. In some embodiments, the method may include providing a printed circuit board comprising electric components. The printed circuit board may be the electric circuit 210 or the electric circuit 1008. The method may then include coupling the cover 206 to the electric circuit 210 or coupling the cover 1016 to the electric circuit 1008. The cover 206 and the cover 1016 may be configured to provide a fluid seal around at least one electronic component of the printed circuit board. The method may further include encapsulating the printed circuit board and the cover in an encapsulant. In some embodiments, this may include encapsulating the electric circuit 210 and the cover 206 in the encapsulant 204 or encapsulating the electric circuit 1008 and the cover 1016 in the encapsulant 1004.

In some embodiments, the method may further include drilling at least one hole through the encapsulant and the cover to provide a fluid path from the at least one electronic component to an exterior of the device. More specifically, one or more holes may be drilled through the encapsulant 204 and the encapsulant 1004 to expose at least one electronic component respectively to an ambient environment surrounding the system 200 or the system 1000.

The systems, apparatuses, and methods described herein may provide significant advantages. For example, the system 200 includes the negative pressure device 202 which may include fewer components and may reduce the number of seals which may improve the likelihood of successful sealing of the system 200. Similarly, the system 1000 include the wound sensing device 1002 which may also result in fewer components and may reduce the number of seals which may improve the likelihood of successful sealing of the system 1000. Both the system 200 and the system 1000 may have improved robustness and reduced overall size which may be desirable to a user of the system 200 or the system 1000.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations the dressing 110, the container 115, or both may be eliminated or separated from other components for manufacture or sale. In other example configurations, the controller 130 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A system for treating a tissue site with negative pressure, the system comprising:
a dressing configured to be disposed at a tissue site;
an electric circuit including a power source and at least one sensor;
a cover including at least one cavity and a negative pressure port, the at least one cavity configured to create a sealed enclosure around the at least one sensor;
an encapsulant surrounding the cover and at least a portion of the electric circuit;
a negative pressure source; and
a tube configured to be fluidly coupled to the negative pressure port and to communicate negative pressure between the negative pressure source and the dressing.

2. The system of claim 1, wherein the cover further comprises a negative pressure cavity in fluid communication with the negative pressure port, and wherein the negative pressure cavity is configured to be positioned in fluid communication with the dressing.

3. The system of claim 2, wherein the cover further comprises a sensing cavity configured to be fluidly isolated from the negative pressure cavity, wherein the sensing cavity is configured to create the sealed enclosure around the at least one sensor, and wherein the at least one sensor is a pressure sensor.

4. (canceled)

5. The system of claim 1, wherein the electric circuit comprises a printed circuit board, and wherein the cover is configured to create the sealed enclosure relative to the printed circuit board.

6. (canceled)

7. The system of claim 5, wherein the printed circuit board is formed of a material that is more rigid than the encapsulant.

8. The system of claim 1, wherein the at least one cavity is a negative pressure cavity configured to create the sealed enclosure around the at least one sensor, and wherein the negative pressure cavity is in fluid communication with the negative pressure port and configured to be positioned in fluid communication with the dressing.

9. The system of claim 1, wherein the electric circuit includes the negative pressure source, wherein the at least one sensor comprises a negative pressure sensor and an ambient pressure sensor, and wherein the cover is further configured to create the sealed enclosure around the negative pressure source and the negative pressure sensor.

10. The system of claim 9, wherein the sealed enclosure is a first sealed enclosure and the cover is further configured to create a second sealed enclosure configured to enclose the ambient pressure sensor separate from the first sealed enclosure.

11. The system of claim 10, wherein the cover comprises:
a first exhaust aperture configured to provide a path for exhaust from the negative pressure source to escape through the cover; and
a first ambient pressure sensor aperture configured to provide a path from the ambient pressure sensor to an ambient environment surrounding the system.

12. The system of claim 11, wherein the encapsulant comprises:
a second exhaust aperture configured to provide a path for exhaust from the negative pressure source to escape through the first exhaust aperture and the encapsulant; and
a second ambient pressure sensor aperture configured to provide a path from the ambient pressure sensor through the first ambient pressure sensor aperture to the ambient environment surrounding the system.

13. The system of claim 12, further comprising:
a first filter configured to couple with the second exhaust aperture;
a second filter configured to couple with the second ambient pressure sensor aperture;
a membrane configured to couple to the encapsulant to enclose the first filter and the second filter between the membrane and the encapsulant; and
an ambient environment pathway on a surface of the encapsulant, the ambient environment pathway configured to connect the second exhaust aperture and the second ambient pressure sensor aperture to the ambient environment surrounding the system.

14. The system of claim 9, wherein the encapsulant comprises:
an ambient pressure sensor aperture configured to provide a path from the ambient pressure sensor to an ambient environment surrounding the system; and
a first exhaust aperture configured to align with a second exhaust aperture of the cover, the first exhaust aperture and the second exhaust aperture configured to provide a path for exhaust from the negative pressure source to reach the ambient environment surrounding the system.

15. The system of claim 9, further comprising a power button configured to actuate the negative pressure source, wherein the power button is embedded within the encapsulant, and wherein the encapsulant is configured to allow a user to depress the power button.

16. (canceled)

17. The system of claim 1, wherein the encapsulant comprises a power source aperture configured to expose the power source to an ambient environment surrounding the system.

18. The system of claim 17, wherein the at least one sensor comprises an ambient pressure sensor disposed proximate to the power source such that the ambient pressure sensor and the power source are both exposed to the ambient environment surrounding the system through the power source aperture.

19. (canceled)

20. The system of claim 1, wherein the at least one cavity comprises a negative pressure cavity and a sensing cavity isolated from the negative pressure cavity, wherein the at least one sensor comprises a negative pressure sensor and an ambient pressure sensor, wherein the negative pressure cavity is configured to create a first sealed enclosure around the negative pressure sensor, wherein the sensing cavity is configured to create a second sealed enclosure around the ambient pressure sensor, wherein the first sealed enclosure is fluidly isolated from the second sealed enclosure, and wherein the negative pressure cavity is in fluid communication with the negative pressure port and configured to be positioned in fluid communication with the dressing.

21. The system of claim 1, wherein the at least one sensor comprises a negative pressure sensor and an ambient pressure sensor, wherein the at least one cavity of the cover comprises a sensing cavity fluidly isolated from a negative pressure cavity, wherein the sensing cavity is configured to create the sealed enclosure around the negative pressure sensor, wherein the sensing cavity and the sealed enclosure are fluidly isolated from the ambient pressure sensor, and wherein the negative pressure cavity is in fluid communication with the negative pressure port.

22. The system of claim 21, wherein the cover further comprises:
a sensing aperture configured to expose the sensing cavity and the negative pressure sensor to the dressing; and
a negative pressure opening configured to provide a path from the negative pressure cavity to the dressing.

23. The system of claim 22, wherein the encapsulant comprises an opening configured to couple the sensing aperture and the negative pressure opening to the dressing.

24. (canceled)

25. (canceled)

26. (canceled)

27. A method of manufacturing a device comprising:
providing a printed circuit board comprising electronic components;
coupling a cover around at least one electronic component, the cover configured to provide a fluid seal around the at least one electronic component;
encapsulating the printed circuit board and the cover in an encapsulant; and
providing at least one hole through the encapsulant and the cover to provide a fluid path from the at least one electronic component to an exterior of the device.

28. (canceled)

29. (canceled)
